(19) United States
(12) Patent Application Publication
(10) Pub. No.: US 2014/0187987 A1
(43) Pub. Date: Jul. 3, 2014

(54) CUFF FOR ARTERIAL BLOOD PRESSURE MONITOR

(71) Applicants: Jacob Fraden, San Diego, CA (US); Justin Davidson, San Diego, CA (US); William Ewing, Encinitas, CA (US)

(72) Inventors: Jacob Fraden, San Diego, CA (US); Justin Davidson, San Diego, CA (US); William Ewing, Encinitas, CA (US)

(73) Assignee: KAZ USA, INC, Southborough, MA (US)

(21) Appl. No.: 13/730,180

(22) Filed: Dec. 28, 2012

(51) Int. Cl. A61B 5/022

(52) U.S. Cl. CPC A61B 5/02233
USPC 600/499

(57) ABSTRACT

A sphygmomanometer with a cuff for use on a patient wrist, upper or lower arm incorporates an inflatable bladder and a support structure. The cuff is subdivided into two sections. The first section holds the bladder against an arterial side of the limb, while the second section abuts a non-arterial side of the limb and is mechanically coupled to the support structure. When the cuff is attached to the patient limb, the bladder is positioned to avoid receiving a gravitational force caused by the weight of the limb. Rather, the gravitational force is absorbed by the support structure in an interior area of the cuff removed from the bladder.

### CROSS-REFERENCE TO RELATED APPLICATIONS

This application is a continuation of U.S. application Ser. No. 13/505,673, filed May 2, 2012, which was the National Stage of International Application No. PCT/US2009/063972, filed on Nov. 11, 2009. The contents of all of these applications are incorporated herein by reference.

### FIELD OF THE INVENTION

This invention relates generally to methods and medical apparatuses for non-invasive monitoring of arterial blood pressure, and specifically to the devices and methods that use inflatable cuffs.

### BACKGROUND OF THE INVENTION

Blood pressure monitoring has rapidly become an accepted and, in many cases, essential aspect of human and veterinary treatment. Blood pressure monitors are now a conventional part of the patient environment in emergency rooms, intensive and critical care units, in the operating theater, and in homes.

Several well known techniques have been used to non-invasively monitor a subject's arterial blood pressure waveform, namely: auscultation, oscillometry, tonometry and flowmetry. The auscultation, oscillometric and flowmetry techniques use a standard inflatable cuff that occludes an artery (for example, the subject's brachial artery). The auscultatory technique determines the subject's systolic and diastolic pressures by monitoring certain Korotkoff sounds that occur as the cuff is slowly deflated or inflated. The oscillometric technique, on the other hand, determines these pressures, as well as the subject's mean pressure, by measuring the small pressure oscillations that occur in the cuff as the cuff is deflated or inflated. The flowmetric technique relies on detecting variations in blood flow downstream from the cuff

The oscillometric method of measuring blood pressure is currently the most popular method in commercially available automatic systems. This method relies on measuring changes in arterial counter pressure, such as imposed by an inflatable cuff, which is controllably relaxed or inflated. In some cases, the cuff pressure change is continuous, and in others it is incremental. In all oscillometric systems, a transducer (pressure sensor) monitors arterial counter pressure oscillations, and processing electronics convert selected parameters of these oscillations as represented by signals produced by the transducer into blood pressure data.

In the oscillometric method, the mean blood pressure value is the mean of the cuff pressure values that correspond in time to a peak of the envelope of the pressure oscillations. Systolic blood pressure is generally estimated as the pressure of a decaying pressure slope prior to the peak of the pressure oscillations envelope, corresponding to a point in time where the amplitude of the envelope is equal to a fraction of the peak amplitude. Generally, systolic blood pressure is the pressure on the decaying pressure of the cuff prior to the peak of the envelope where the amplitude of the envelope is 0.57 to 0.45 of the peak amplitude. Similarly, diastolic blood pressure is the pressure on the decaying pressure of the cuff after the peak of the envelope that corresponds to a point in time to where the amplitude of the envelope is equal to a different fraction of the peak amplitude. For example, diastolic blood pressure may be conventionally estimated as the pressure on the decaying pressure of the cuff after the peak where the amplitude of the envelope is equal to 0.82 to 0.74 of the peak amplitude. Other algorithms are also well known in the art.

The auscultatory method also involves inflation of a cuff placed around a cooperating artery of the patient. Systolic pressure is indicated when the Korotkoff sounds disappear as the cuff is inflated above the highest pressures exerted by the heart onto the arterial walls. Diastolic pressure is indicated when the Korotkoff sounds first appear as the cuff pressure is elevated above the atmospheric pressure. The auscultatory method can only be used to determine systolic and diastolic pressures, and it does not determine mean pressure.

To use either of the oscillometric and ausculatory methods of arterial pressure computation, an oscillatory signal of sufficient quality must be obtained from the artery. The signal quality (for example, as determined by pulse shape distortion and noise level) is greatly influenced by a matching between the inflatable cuff and the patient limb geometry. The cuff size should correspond to the length and circumference of the limb. A fluid bladder positioned inside the cuff should be wrapped around at least a portion of the limb in such a manner as to fully envelop the arterial path, and to effect a gradual and full compression of the artery when pressure in the bladder reaches the systolic pressure inside the artery. The pressure generated by the cuff should not be affected by a gravitational force exerted by the weight of the limb. In other words, the bladder should not compressed by any external forces except the fluid pump and the arterial blood pressure. In addition, when the cuff is positioned on or near the wrist, the wrist should be elevated approximately at the aorta level, otherwise a hydrostatic pressure of blood will cause additional errors. Generally speaking, with consideration of the above-described objectives, prior art pressurizing cuffs have had the following deficiencies: a need for a manual adjustment of the cuff size to match the limb size, and deleterious effects caused by hydrostatic pressure and the limb weight on the accuracy of the pressure measurement.

To minimize errors that arise from the above deficiencies, numerous cuff designs have been proposed. U.S. Pat. No. 3,527,204 to Lem, which is incorporated by reference herein in its entirety, discloses a dual cuff having a liquid-filled chamber positioned on the top of an air-filled chamber, configured so that the pressure exerted over a patient's limb is developed by applying pressure to both air and liquid. A dual-cuff design with side-by-side bladders is described in U.S. Pat. No. 3,752,148 to Schmalzbach, which is incorporated by reference herein in its entirety. A dual air chamber cuff design with two chambers positioned in layers is disclosed in U.S. Pat. No. 7,250,030 to Sano et al., which is incorporated by reference herein in its entirety. A cuff designed with a semi-rigid outer layer on an outside surface of the cuff is described in U.S. Pat. No. 6,224,558 to Clemmons, which is incorporated by reference herein in its entirety. U.S. Pat. No. 6,336,901 to Itonaga et al., which is incorporated by reference herein in its entirety, discloses a cuff design including two air bags that are sequentially inflated to provide for a more uniform arterial compression.

Other cuff designs have been proposed to improved the manner in which the cuff is initially fit over a patient's limb. See, e.g., U.S. Pat. No. 6,565,524 issued to Itonaga et al. (elastic cuff with elastic plate having a curvature matched to a limb site to be measured), U.S. Pat. No. 7,144,374 to Sano et al. (cuff having adjustable belt applied over a radially changeable elastic member) and U.S. Pat. No. 7,083,573 to Yamakoshi et al. (cuff configured as split ring with pivot), each of which is incorporated by reference herein in its entirety. However, each of the above-referenced cuff designs fails to provide sufficient measurement accuracy. As a result, it would be of benefit to provide a cuff design which can be easily applied to a limb while exhibiting improved measurement accuracy.

### SUMMARY OF THE INVENTION

The present invention is directed to a cuff for a sphygmomanometer that can be used to measure arterial blood pressure from a patient's limb (for example, at a patient's wrist, upper arm or lower arm) while the limb is positioned in a gravitational field. The cuff includes interconnected first and second sections, where the first section is configured to position a pressurizing device (for example, an air bladder) against an arterial side of the patient's limb, while the second section is mechanically coupled to a support. The pressurizing device is coupled with a pressure sensor for monitoring pressure oscillations in the pressurizing device that are indicative of an arterial blood pressure.

The second section and the support are mutually arranged within the gravitational field to direct a vector of the gravitational field away from the arterial side and toward a rear side of the patient's limb, such that substantially no gravitational force is applied to the pressurizing device. In this arrangement, the force generated by the limb within the gravitational field is instead absorbed by the second section and the support. The cuff has a variable geometry that allows the patient's limb to be easily inserted and then fixedly gripped so that it may be supported by the second section. By diverting the effects of gravitational force away from the pressurizing device, a signal-to-noise ratio of the signals provide by the pressure sensor is improved for more accurate blood pressure measurement

### BRIEF DESCRIPTION OF THE DRAWINGS

The foregoing and other features of the present invention will be more readily apparent from the following detailed description and drawings of illustrative embodiments of the invention, in which:

FIG. 1 provides a perspective view of a sphygmomanometer including a measurement cuff according to an embodiment of the present invention;

FIG. 2 provides a cross-sectional view of the sphygmomanometer of FIG. 1;

FIG. 3 provides a front view of another sphygmomanometer including a measurement cuff according to an embodiment of the present invention;

FIG. 4 provides a side view of the sphygmomanometer of FIG. 3;

FIG. 5 provides a side view of another sphygmomanometer including a measurement cuff according to an embodiment of the present invention;

FIG. 6 provides a perspective view of a variant to the sphygmomanometer of FIG. 4 including a measurement cuff according to an embodiment of the present invention;

FIG. 7 provides a perspective view of another sphygmomanometer including a measurement cuff according to an embodiment of the present invention;

FIG. 8 is a cross-sectional view of the sphygmomanometer of FIG. 7;

FIG. 9 provides a perspective view of another sphygmomanometer including a measurement cuff according to an embodiment of the present invention; and

FIG. 10 a cross-sectional view sphygmomanometer of FIG. 9.

Like reference numerals are used in the drawing Figures to connote like components of the sphygmomanometer and measurement cuff.

### DETAILED DESCRIPTION OF THE PREFERRED EMBODIMENTS

The present invention relates to non-invasive arterial blood pressure measurement methods using pressurizing cuffs with suitable pressurizing devices (for example, inflatable bladders). Pressure inside the bladder may be generated by a compressed fluid. For example, the compressed fluid may be selected to be air that is compressed and provided to the bladder by a conventional air pump and released from the bladder by a conventional decompression valve). The pressure generated by the bladder is preferably monitored using a pressure sensor coupled to the bladder.

The oscillometric method described above may be performed by analyzing oscillations in cuff pressure measurements caused by blood surges passing through a pliant artery that transmit pressure pulses to the bladder. The auscultatory method described above may be performed by analyzing the characteristics of acoustic waves (Korotkoff sounds) produced inside the compressed artery. In each case, embodiments of the method rely on accurate detection of the mechanical oscillations or vibrations of the artery that are of arteries that are transmitted to the bladder.

These oscillations and vibrations may be detected by a corresponding sensor coupled to the bladder. One source of error operating when a conventional cuff is wrapped around a patient's wrist and positioned on a tabletop is the weight of the arm and hand. Even small variations in the gravitational force can result in spurious oscillations and vibrations inside the cuff, and thereby contaminate the signals indicating oscillations and vibrations from the arteries. For example, such pressure variations may be caused by patient motions or external vibrations (generated, for example, when the patient is being transported). To minimize such spurious signals, embodiments of the present invention rely on a combination of two design features: a decoupling of the inflatable cuff from the support structure, and a cuff geometry that is adjusted for the size and shape of the patient limb. A key idea behind preferred embodiments of the invention is decoupling the gravitational force from the arterial side of the limb, and directing it toward a back side of the cuff that is adjacent to the rear side of the limb.

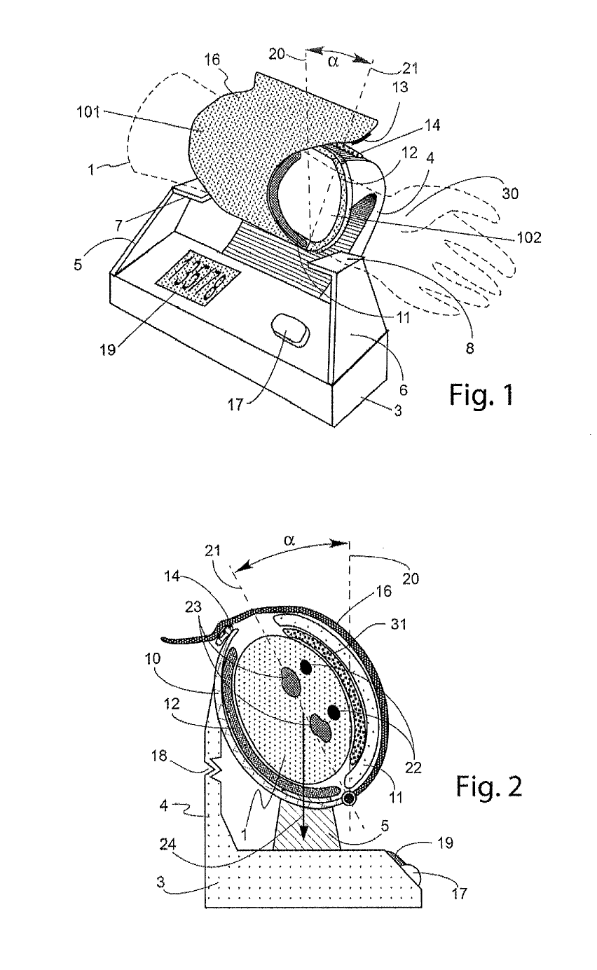

FIG. 1 illustrates a sphygmomanometer according to an embodiment of the present invention that includes a cuff 16 divided into two sections. A first section 101 contains a bladder 11, and a second section 102 comprises a back support 10 supported by a stem 4. The cuff 16 is wrapped around a patient's limb 1, and locked in place with a suitable locking device such as a locking tape 13, 14 (for example, a hook and loop fastener such as a VELCRO® fastener). An inflatable bladder 11 is positioned on the inner side of the cuff 16 (within the boundaries of the first section 101) to face an inner side of a wrist of the patient's limb 1. In other words, the first section 101 is configured to face the arterial side of the patient's limb. The bladder is preferably formed from an elastic material, such as latex, synthetic or natural, or any elastomeric material, such as polyurethane.

The sphygmomanometer of FIG. 1 further includes an electronic module that is incorporated inside a base 3, a display 19 and at least one control button 17. The back support 10 preferably includes a cushion 12 to comfortably support the patient's limb 1 against the back support 10. The bladder 11 is inflatable to compress arteries inside the limb 1, causing a restriction of the blood flow inside the arteries. The restriction generates arterial oscillations which can be detected by a conventional pressure sensor or accelerometer coupled to the bladder 11.

As illustrated for example in FIG. 2, the back support 10 can be attached to a base 3 by a stem 4. During operation, the base 3 is preferably placed on a platform such as a tabletop. Referring again to FIG. 1, two armrests 7, 8 are coupled to the base 3 by corresponding stand-offs 5 and 6. The armrests 7, 8 support the patient limb 1 at positions away from the first section 101. By supporting the weight of the limb 1 during blood pressure monitoring, the armrests 7, 8 relieve the cuff 16 from supporting the full weight of the limb 1 and assist in reducing the effect of the weight of the limb 1 on signal noised generated at the pressurizing device.

Besides the pressure sensor, base 3 may contain other components, such as a power supply, other sensors, electronic circuitry, an internal pump, valves, and the like. A hose assembly for connecting the bladder 11 to the internal pump, pressure sensors and valves may preferably be hidden inside the base 3 and stem 4. A liquid-filled bag 31 as shown in FIG. 2 may preferably be provided at a position between the bladder 11 and limb 1 to improve pressure compliance with the arterial blood flow.

The sphygmomanometer of FIGS. 1 and 2 may be operated as follows. Initially, as locking tape 13, 14 is unlocked, the first section 101 of the cuff 16 is released from the back plate 10, and the limb 1 (a patient's arm, as illustrated in FIG. 1) is placed on the cushion 12 in a manner such that a wrist 30 faces outwardly to position an inner surface (arterial side) of the limb 1 outwardly such that arteries 22 are positioned away from the cushion 12. The cuff 16 is then wrapped around the limb 1, and the locking tape 13, 14 is secured. In this configuration, the bladder 11 and liquid-filled bag 31 (if provided) face the arteries 22.

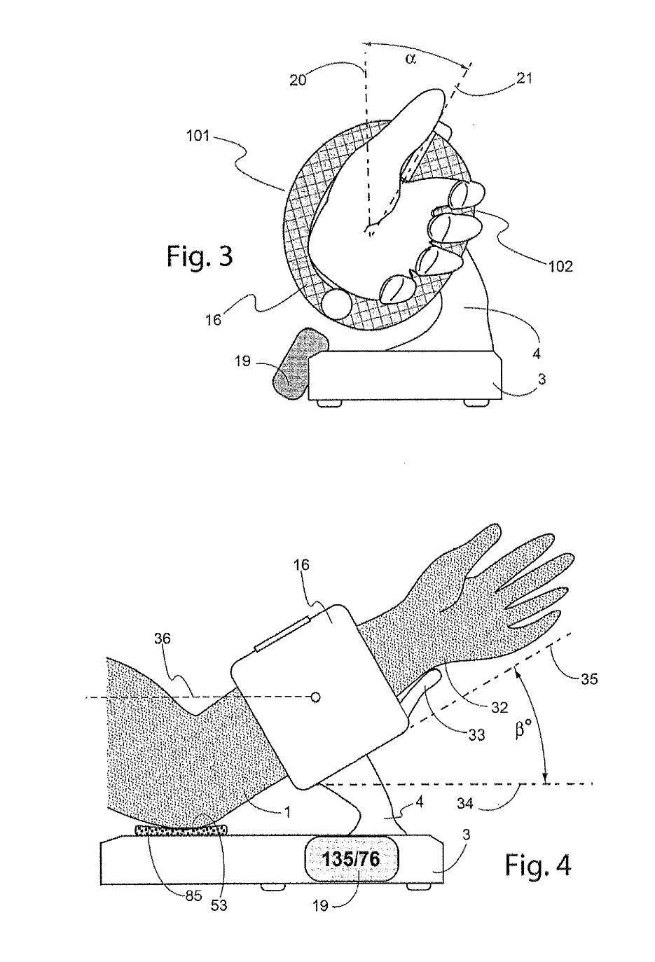

An operator proceeds to press a switch 17, which initiates a measurement cycle of the sphygmomanometer. The internal pump pressurizes the bladder 11 to compress the arteries 22 against supporting bones 23 inside the limb 1. As illustrated in FIG. 2, an axis 21 of the back plate 10 is tilted by an angle α with respect to a vertical direction 20 of the sphygmomanometer. The base 3 of the sphygmomanometer is preferably positioned so that the vertical direction 20 is parallel to a gravity vector 24. Because the limb 1 in this configuration is primarily supported by the cushion 12 and back 10, the gravitational force vector 24 is directed toward the support 5, and away from bladder 11 and the arterial side of the limb 1. The angle α should preferably be set between 20° and 60° (see also FIG. 3).

The bladder 11 receives arterial oscillations from the arterial side of the limb 1, and transmits the oscillations to the internal pressure sensor. In response, the internal pressure sensor transmits a signal to the electronic circuit, and the electronic circuit translates the signal to determine a pressure inside the bladder 11, to compute systolic and diastolic pressure values, and to transmit signals to the display 19 for displaying the systolic and diastolic pressure values. Since the gravity vector 24 is directed away from the bladder 11, distortions in the arterial pressure arising from variations in the weight vector 24 (for example, as would arise from movements by the patient of the limb 1) are reduced. As illustrated in FIG. 2, the stem 4 may preferably incorporate a pivot and/or spring 18 configured to further absorb variations in the gravity vector 24 due to patient movement of the limb 1 while it supported by armrests 7, 8.

To minimize effects of hydrostatic pressure generated by the weight of the blood fluid, it is desirable to elevate the cuff approximately to a vertical level 36 substantially equal to the vertical level of an aorta of the patient. In an embodiment of the present invention as illustrated in FIG. 4, the stem 4 is configured to tilt the cuff 16 with respect to a horizon 34 to form an angle β between the horizon 34 and a cuff axis 35. A horizontal plane defined by the horizon 34 is perpendicular to the direction of the gravity vector. By tilting the cuff 16 in this manner, it can be positioned in proximity to the level 36.

Further, to set the cuff 16 at a predetermined position in relation to the wrist 3 of the limb 1, a guide 33 is preferably provided. When the limb 1 is held by the cuff 16, the guide 33 is configured to rest at the base 32 of the patient's thumb, thereby setting a longitudinal position of the cuff 16 relative to the patient's wrist 30. In this manner, the guide 33 positions the cuff 16 consistently, thereby improving repeatability of successive blood pressure measurements. A pillow 85 is preferably provided on the base 3 for supporting an elbow 53 of the limb 1 in a comfortable and stable manner.

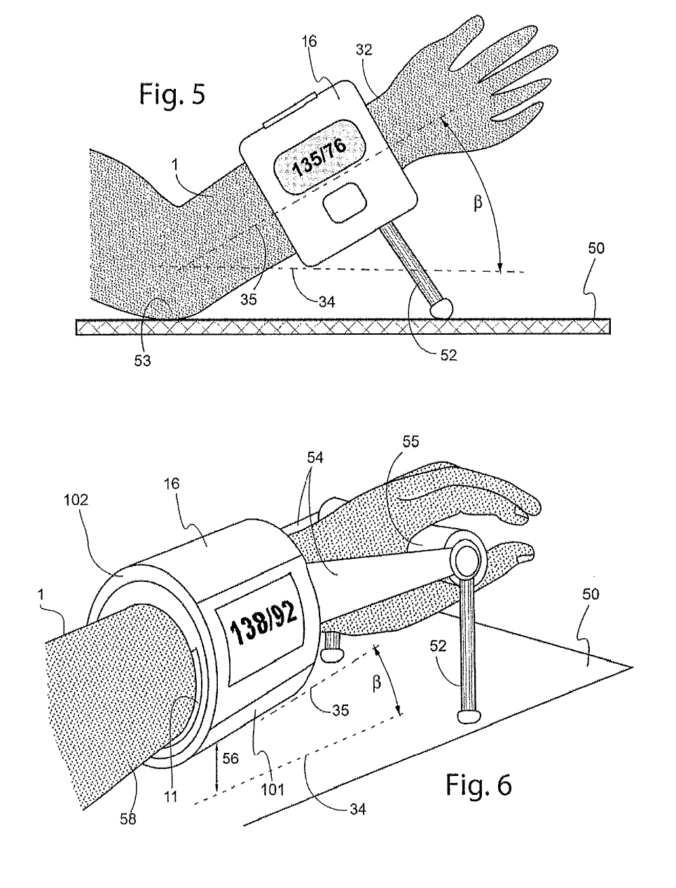

Alternate configurations for tilting and supporting the limb 1 to relieve the bladder 11 from the gravity vector 24 are illustrated in FIGS. 5 and 6. Both configurations employ one or more legs 52 that may be positioned to rest on and against a tabletop 50 to support the sphygmomanometer and the limb.

As illustrated in FIG. 6, the effect of the gravity vector 24 can be further isolated from the bladder 11 by providing links 54 and a hand rest 55 for further stabilizing the position of the wrist 30 of the limb 1 in relation to the cuff 16. The links 54 preferably comprise a flexible material (for example, nylon or another suitable plastic) to further absorb variations in the gravity vector 24 due to patient movement of the limb 1. As shown in FIG. 5, an axis 51 of the limb 1 is tilted with respect to a horizon 34 by an angle β that is preferably set between 20 and 45°. As previously described, this positioning helps to keep the level of the cuff 16 approximately at the level of the aorta, and away from tabletop 50 by a distance 56 to safely ensure that the cuff 16 makes no contact with the tabletop 50 during use to negatively affect measurement accuracy. In this configuration, an inner part 58 of the limb 1 (artery side) and the bladder 11 are accordingly not affected by the weight of the limb 1.

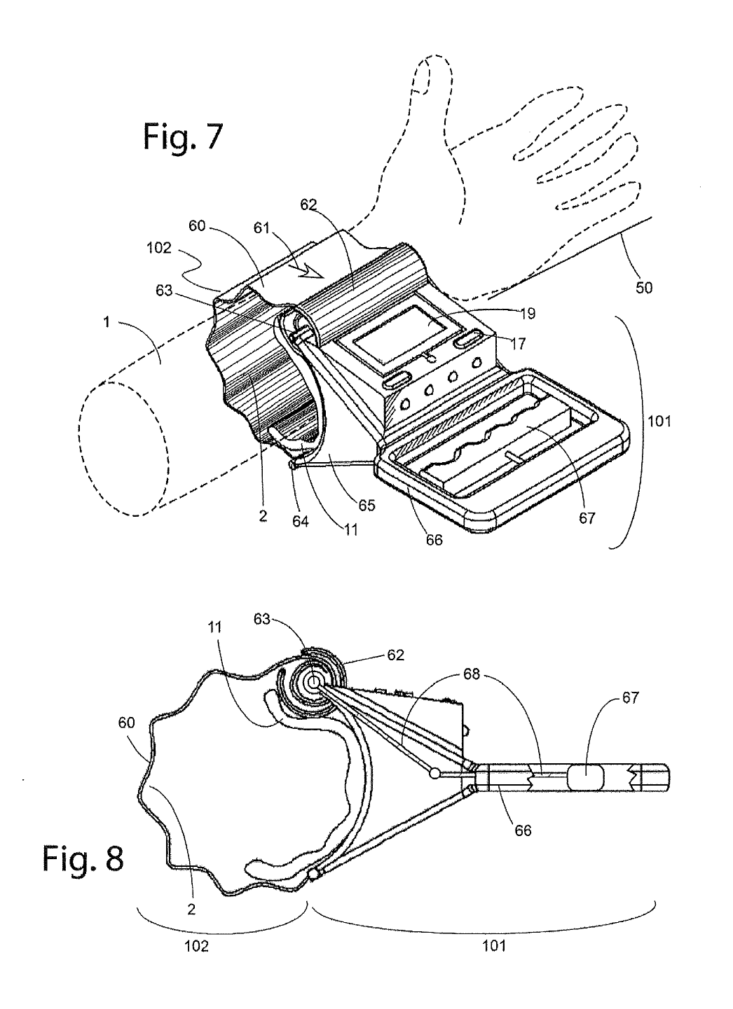

In addition to relieving the bladder 11 from effects of the gravity vector 24, the cuff 16 may be sized to provide good compliance in gripping the limb 1. In other words, the limb 1 is preferably well-supported by the cuff 16, while at the same time decoupling the weight of the cuff 16 from the bladder 11. Thus, a rear side of the limb 1 (away from the arteries) is preferably not mechanically coupled to the bladder 11, but instead is coupled to a weight-supporting part of the cuff. This is illustrated for example in FIG. 7, which illustrates a sphygmomanometer according to another embodiment of the present invention.

The sphygmomanometer of FIG. 7 contains a base 65 that supports the bladder 11, and is coupled with a retractable belt 60 that is soft and pliant (for example, a rubberized woven fabric). The belt 60 may preferably be retractably rolled onto a spool 63 rotatably provided within a holding cylinder 62. The spool 63 is preferably spring-loaded to retract the belt 60 within the spool 63 under the control of a grip 67 positioned inside a handle 66. The handle 66 serves as a support for the sphygmomanometer, and is in effect a functional equivalent to the support 4 of FIGS. 1 and 2. During operation, it is held by an operator in order to support the weight of the limb 1 against the belt 60.

As illustrated in FIGS. 7 and 8, one end of the retractable belt 60 is fixed to a pin 64, while the opposite end is attached to the spool 63 so that the belt 60 is movable in a direction 61 into the cylinder 62 until the retractable belt 60 fully embraces the limb 1. In operation, the operator squeezes the grip 67 which, via links 68, operates the spool 63 to release and allow the retractable belt 60 to expand outwardly from the cylinder 62. The limb 1 (for example, beginning with the patient's hand as illustrated in FIG. 7) can be inserted through the expanded retractable belt 60. At this time, the bladder 11 is deflated and the pressure sensor coupled to the bladder 11 is “zeroed” with respect to atmospheric pressure. Next, the operator releases the grip 67, and the spool 63 rotates under spring force to pull the retractable belt 60 in the direction 61 until it tightly encircles the limb 1. The spool 63 preferably includes a ratchet or other conventional “one-way” mechanism, causing the tightened belt 60 to become locked such that it can no longer be tightened or expanded without further squeezing the grip 67. An air pump preferably provided within the base 65 inflates the bladder 11, and arterial pressure is measured by one of the previously-described, known methods known in art. The weight of the limb 1 is supported by the back side 2 of the tightened belt 60 and, subsequently, by handle 66, while the arterial side of the arm is exposed only to pressure exerted by the bladder 11 and not exerted by the weight of the limb 1. The weight of the limb 1 may be further supported by resting the limb 1 on a side of the tabletop 50, or by using one of the supporting structures shown in FIGS. 1-6.

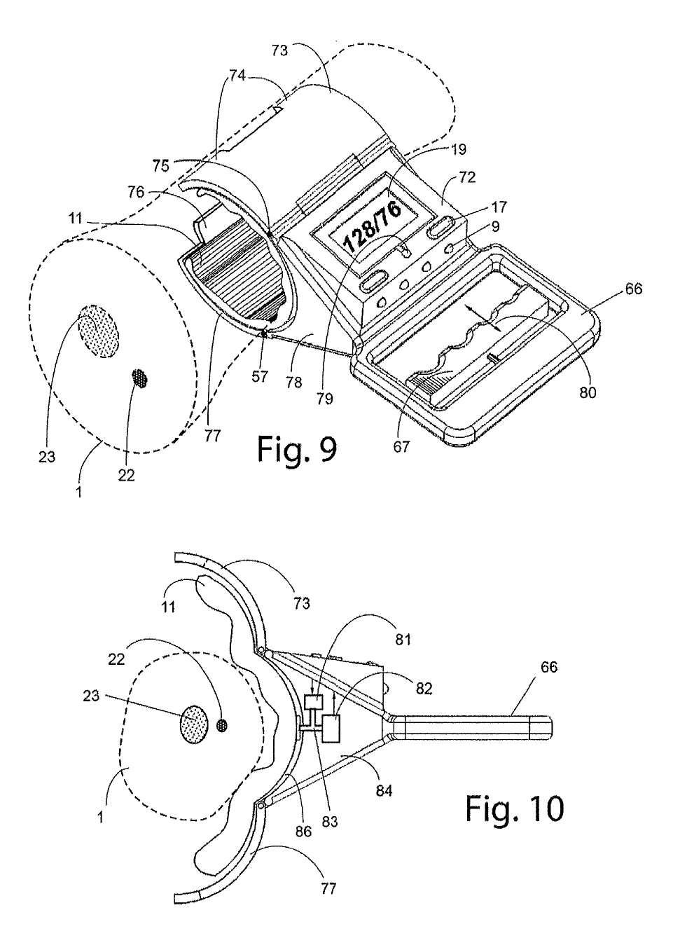

An alternative embodiment of the cuff 16 of FIGS. 7 and 8 is shown in FIGS. 9 and 10. In the embodiment of FIGS. 9 and 10, the retractable belt is replaced by an articulated, three-part jaw including a base 78 and clamps 73, 77 which are rotatably coupled to the base 78 by pivots 75 and 57, respectively. The bladder 11 is configured so that it does not protrude beyond open ends of the clamps 73 and 77. When the clamps 73 and 77 close, they support the limb 1 at lips 74 and 76, respectively, so that the bladder 11 is relieved from supporting the arm's weight once the cuff 16 is rotated counter-clockwise to its position as shown in FIGS. 9 and 10.

The clamps 73 and 77 can be opened by squeezing the grip 67 to move in a direction 80. When the grip 67 is squeezed, the clamps 73 and 77 open so that the bladder 11 may be positioned against the arterial side of the limb 1 in proximity to an interior surface 86 of the base 78. In this position, the artery 22 can be compressed by the bladder 11 against the bone 23. Once the bladder 11 is so positioned, the grip 67 is released, the clamps 73, 77 are rotated to close and tightly encircle the limb 1. To facilitate closure, the clamps 73, 77 are preferably provided with conventional spring-return mechanisms.

As illustrated in FIG. 10, an internal pump 81 controlled by an electronic control circuit may be housed within an internal cavity 84 of the base 78 of the sphygmomanometer, and inflates the bladder via an inflation tube 83. A pressure sensor 82 in communication with the bladder 11 via the inflation tube 83 can sense a bladder pressure, and transmits a signal indicating the bladder pressure to the electronic control circuit for processing. The electronic control circuit is preferably housed within a control panel 72 of the sphygmomanometer.

As illustrated in FIG. 10, the control panel 72 may include one or more control buttons 17 for activating the electronic circuit, pump 81, pressure sensor 82 and electronic control circuit, and is preferably equipped with a reset button 79 to clear the display and reset the sphygmomanometer for a subsequent measurement. The control panel 72 is also preferably equipped with indicator lamps 9 for providing an indication of a current status of the arterial blood pressure measurement.

While the invention has been particularly shown and described with reference to a number of preferred embodiments thereof, it will be understood by those skilled in the art that various changes in form and details may be made therein without departing from the spirit and scope of the invention. Accordingly, the invention is to be limited only by the scope of the claims and their equivalents.

What is claimed is:

1. A cuff for a non-invasive measurement of an arterial blood pressure from a patient's limb, such limb having an arterial side and a rear side and being positioned in a gravitational field, the cuff comprising:
interconnected first and second sections, wherein the first section comprises a pressurizing device configured to be pressurized by a pressure source, the pressurizing device being configured for positioning adjacent to the arterial side of the limb; and
a support mechanically coupled to the second section, wherein the second section and the support are mutually arranged within the gravitational field to direct a vector of the gravitational field in a direction away from the arterial side and toward the rear side of the patient's limb, such that substantially no gravitational force is applied to the pressurizing device.

2. The cuff of claim 1, wherein the pressurizing device comprises a flexible bladder configured to be pressurized with a fluid.

3. The cuff of claim 2, further comprising a pressure sensor coupled to the bladder.

4. The cuff of claim 2, further comprising a cushion provided inwardly from the bladder for positioning between the bladder and the limb.

5. The cuff of claim 2, further comprising a cushion provided inwardly from the second section for positioning between the second section and the limb.

6. The cuff of claim 1, wherein the support further comprises:
a base configured for positioning the support against a fixed surface; and
a stem that interconnects the second section to the base.

7. The cuff of claim 6, wherein the stem further comprises a pivot member configured to absorb variations in the gravity vector due to movement of the limb.

8. The cuff of claim 7, wherein the pivot member comprises a spring.

9. The cuff of claim 6, further comprising:
one or more rests coupled to the base and positioned externally and away from the first section for providing additional support to the limb.

10. The cuff of claim 1, wherein the first section is pivotally connected to the second section at a first end, and is adjustably and removably fixed to the second section at a second end.

11. The cuff of claim 10, wherein the first section is adjustably and removably fixed to the second section via a hook and loop fastener.

12. The cuff of claim 1, wherein the support is configured for positioning an axis between the first and second sections at an angle α from a direction of the vector of the gravitational field, wherein the angle α is set within a range of 20° to 60°.

13. The cuff of claim 1, wherein the support comprises at least one leg mechanically coupled to the second section, the at least one leg being configured for positioning against a fixed surface.

14. The cuff of claim 1, further comprising:
a hand rest mechanically coupled to the second section; and
a plurality of legs mechanically coupled to the hand rest, wherein:
the plurality of legs are configured for positioning against a fixed surface, and
the hand rest is configured for gripping by a hand of the patient in order to stably position a wrist of the patient along a longitudinal axis of the cuff.

15. The cuff of claim 1, wherein the support is configured for positioning a longitudinal axis of the cuff at an angle β from a horizontal plane that is perpendicular to the vector of the gravitational field, wherein the angle β is set within a range of 20° to 45°.

16. The cuff of claim 1, further comprising:
a guide extending externally from the cuff, the guide being configured to abut a feature of the limb in order to stably position the limb along a longitudinal axis of the cuff.

17. The cuff of claim 16, wherein the guide is configured to abut a base of the patient's thumb.

18. The cuff of claim 1, further comprising at least one of a display and a control button.

19. The cuff of claim 1, wherein the second section is configured to provide an opening in the cuff that can be expanded and contracted for receiving and gripping the patient's limb.

20. The cuff of claim 19, wherein the support includes a control operable for manipulating the second section to provide an expanded or contracted opening.

21. The cuff of claim 20, wherein the control is operated via a squeezable hand grip.

22. The cuff of claim 20 wherein the second section comprises:
a flexible belt configured to be extendible or retractable for respectively providing an expanded or contracted opening.

23. The cuff of claim 22, further comprising a holding cylinder for receiving a retractable portion of the belt.

24. The cuff of claim 19 wherein the second section comprises:
first and second clamping members pivotally connected to opposing ends of the first section, the first and second clamping members being coordinatedly pivotable with reference to the first section to provide an expanded or contracted opening.

25. A sphygmomanometer comprising:
a cuff for a non-invasive measurement of an arterial blood pressure from a patient's limb, the limb having an arterial side and a rear side and being positioned in a gravitational field, the cuff comprising:
interconnected first and second sections, wherein the first section comprises a pressurizing device configured to be pressurized by a pressure source, the pressurizing device being configured for positioning adjacent to the arterial side of the limb; and
a support mechanically coupled to the second section, wherein the second section and the support are mutually arranged within the gravitational field to direct a vector of the gravitational field in a direction away from the arterial side and toward the rear side of the patient's limb, such that substantially no gravitational force is applied to the pressurizing device.

26. A method of making a measurement of arterial blood pressure from a patient's limb using a sphygmomanometer including a cuff, wherein the limb has an arterial side and a rear side and is positioned in a gravitational field, and wherein the cuff comprises:
interconnected first and second sections, wherein the first section comprises a pressurizing device configured to be pressurized by a pressure source; and
a support mechanically coupled to the second section;
the method comprising the steps of:
positioning the patient's limb inside the cuff with the pressurizing device positioned adjacent to the arterial side of the limb;
rotating the cuff so that the second section and the support are mutually arranged within the gravitational field to direct a vector of the gravitational field in a direction away from the arterial side and toward the rear side of the patient's limb such that substantially no gravitational force is applied to the pressurizing device; and
monitoring a response indicative of an arterial blood pressure at a pressure sensor coupled to the pressurizing device as a pressure in the pressurizing device is increased or decreased.

27. The method of claim 26, wherein the cuff is rotated to a position where an axis between the first and second sections is positioned at an angle α from a direction of the vector of the gravitational field, wherein the angle α is set within a range of 20° to 60°.

28. The method of claim 26, further comprising the step of:
positioning a longitudinal axis of the cuff at an angle β from a horizontal plane that is perpendicular to the vector of the gravitational field, wherein the angle β is set within a range of 20° to 45°.
